SEQUENCE LISTING (12 sequences)

SEQ ID NO: 1 - protein, 110 aa, Artificial Sequence
  anti-Nodal antibody light chain
DIKMTQSPASLSASVGETVTITCRASGNIHNYLAWYQQKQGKSPQLLVYNAKTLADGVPSRFSGSGSGTQYSLKINSLQPEDFGSYYCQHFWSTPHVRCWDQAGTETEAW

SEQ ID NO: 2 - protein, 122 aa, Artificial Sequence
  anti-Nodal antibody heavy chain
  misc_feature (120)..(120): Xaa can be any naturally occurring amino acid
VKLVESGGGLVKPGGSLKLSCAASGFTFSSYAMSWVRQTPEKRLEWVASISSGGCTYYPDSVKGRFTISRDNARNILYLQMSSLRSEDTAMYYCARGSMITADGNSLLLCYGLLGSRNLXHR

SEQ ID NO: 3 - protein, 110 aa, Artificial Sequence
  anti-Nodal antibody heavy chain C
KLVESGGGLVQPGGSMKLSCVASGFTFRNYWMSWVRQSPEKGLEWVAEIRLKSDNYAARYAESVKGKFTISRDDSKSRLYLQMNSLRAEDTGIYYCTGIRRFAYWGQGTL

SEQ ID NO: 4 - protein, 8 aa, Artificial Sequence
  CDRL1
GNIHNYLA

SEQ ID NO: 5 - protein, 11 aa, Artificial Sequence
  CDRL2
LLVYNAKTLAD

SEQ ID NO: 6 - protein, 15 aa, Artificial Sequence
  CDRL3
QHFWSTPHVRCWDQA

SEQ ID NO: 7 - protein, 9 aa, Artificial Sequence
  CDRH1
FTFSSYAMS

SEQ ID NO: 8 - protein, 13 aa, Artificial Sequence
  CDRH2
WVASISSGGCTYY

SEQ ID NO: 9 - protein, 11 aa, Artificial Sequence
  CDRH3
ARGSMITADGN

SEQ ID NO: 10 - protein, 9 aa, Artificial Sequence
  heavy chain C CDRH1 sequence
FTFRNYWMS

SEQ ID NO: 11 - protein, 16 aa, Artificial Sequence
  heavy chain C CDRH2 sequence
WVAEIRLKSDNYAARY

SEQ ID NO: 12 - protein, 7 aa, Artificial Sequence
  heavy chain C CDRH3 sequence
GIRRFAY

(19) United States
(12) Patent Application Publication
(10) Pub. No.: US 2020/0239561 A1
(43) Pub. Date: Jul. 30, 2020

(54) NODAL REGULATION OF CANCER DRUG RESISTANCE GENE

(71) Applicant: TaiRx Inc., Taipei City (TW)

(72) Inventors: Mary J. C. HENDRIX, Shepherdstown, WV (US); Richard E. B. Seftor, Morgantown, WV (US); Elisabeth A. Seftor, Morgantown, WV (US)

(21) Appl. No.: 16/747,419

(22) Filed: Jan. 20, 2020

Related U.S. Application Data
(60) Provisional application No. 62/797,270, filed on Jan. 26, 2019.

(51) Int. Cl. C07K 16/22; A61K 31/337; C07K 16/32; A61K 31/555; A61K 31/704; A61K 31/675; A61P 35/04; G01N 33/574

(52) U.S. Cl. CPC C07K 16/22; A61K 31/337; C07K 16/32; A61K 31/555; G01N 2800/52; A61K 31/675; A61P 35/04; G01N 33/574; G01N 2800/7028; A61K 31/704

(57) ABSTRACT

The invention relates to combination therapeutic agents comprising an anti-cancer drug and an anti-Nodal agent for use in decreasing, suppressing and/or abrogating manifestation or appearance of cancer drug resistance in a cancer subject in need thereof. The anti-Nodal agent decreases, suppresses and/or abrogates cancer drug resistance to the anti-cancer drug in the cancer subject. A pharmaceutical composition comprising an anti-Nodal agent for use in decreasing, suppressing and/or abrogating manifestation or appearance of cancer drug resistance in a cancer subject in need thereof is disclosed. A method for prognosis of cancer patient and a diagnostic kit are also disclosed.

### FIELD OF THE INVENTION

The present invention relates generally to anti-Nodal for therapeutic use in decreasing cancer drug resistance, and more specifically to an anti-Nodal agent for use in reducing expression of the drug resistance gene ABCA1 in a cancer patient.

### BACKGROUND OF THE INVENTION

Poorly differentiated, aggressive breast cancer cells have been shown to possess stem cell properties, proliferate indefinitely, and propagate a tumor comprised of heterogeneous cell subpopulations with varying degrees of drug resistance and metastatic properties [6]. One of the most daunting challenges we face in the oncological sciences is developing the most effective targeting of these heterogeneous tumors containing breast cancer cells expressing various markers, especially those associated with stem cells—for which targeted therapies are currently under development. Particularly noteworthy is the phenotype associated with aggressive triple-negative breast cancer (TNBC), which exhibits little-to-no expression of classical markers, and patients are at significantly higher risk of relapsing with metastatic disease following treatment with standard-of-care therapies. Thus, there is a critical need to identify novel, targetable molecules that can enhance current therapies by mitigating (or neutralizing) the stem cell phenotype of aggressive breast cancer and concurrently reversing drug resistance.

### SUMMARY OF THE INVENTION

In one aspect, the invention relates to use of combination therapeutic agents comprising an anti-cancer drug and an anti-Nodal agent in the manufacture of a medicament for decreasing, suppressing and/or abrogating manifestation or appearance of cancer drug resistance in a cancer subject in need thereof, wherein the cancer subject in need thereof has cancer with heterogeneous tumor cells, in which a subpopulation expresses ATP-binding cassette transporter AI (ABCA1).

In one embodiment, the tumor cell subpopulation expresses Nodal in addition to expressing the ABCA1.

In another embodiment, the tumor cell subpopulation is resistant to the anti-cancer drug.

In one aspect, the invention relates to use of a pharmaceutical composition comprising an anti-Nodal agent in the manufacture of a medicament for decreasing, suppressing and/or abrogating manifestation or appearance of cancer drug resistance in a cancer subject in need thereof, wherein the cancer subject in need thereof has cancer with heterogeneous tumor cells comprising a subpopulation that expresses ATP-binding cassette transporter AI (ABCA1).

In one embodiment, the pharmaceutical composition further comprises an anti-cancer drug to form combination therapeutic agents comprising the anti-Nodal and the anti-cancer drug.

In another embodiment, the cancer is selected from the group consisting of breast cancer, melanoma, prostate cancer, pancreatic cancer and ovarian cancer.

In another embodiment, the cancer subject in need thereof is a recipient of an anti-cancer drug.

In another embodiment, the use of the invention further comprising use of an anti-cancer drug in the manufacture of a medicament for the treatment of the cancer subject in need thereof.

In another embodiment, the use of the pharmaceutical composition comprising the anti-Nodal agent is subsequent to the use of the anti-cancer drug, wherein the tumor cell subpopulation is resistant to the anti-cancer drug.

In another embodiment, the use of the anti-Nodal agent is simultaneous with the use of the anti-cancer drug, wherein the tumor cell subpopulation is resistant to the anti-cancer drug.

In another embodiment, the anti-Nodal agent is an anti-Nodal antibody or CVM-1125.

In another embodiment, the cancer is metastatic cancer.

In another embodiment, the anti-cancer drug is a combination of therapeutic agents selected from the group consisting of (i), (ii) and (iii):
(i) docetaxel, carboplatin, tratuzumab, pertuzumab (TCHP);
(ii) anthracyclines and taxanes (ACT); and
(iii) docetaxel and cyclophosphamide (TC).

In another aspect, the invention relates to a diagnostic kit for use in prognosis of a cancer patient, the diagnostic kit comprising:
(a) an anti-ATP-binding cassette transporter AI (anti-ABCA1) antibody;
(b) an anti-Nodal antibody; and
(c) optionally an instruction sheet for direction of use thereof;
wherein:
(1) the anti-ABCA1 antibody is for use in detecting and/or measuring the level of ATP-binding cassette transporter AI (ABCA1) expression in a tissue sample from the cancer patient;
(2) the anti-Nodal antibody is for use in detecting and/or measuring the level of Nodal in a tissue sample from the cancer patient;
(3) the cancer patient is a recipient of combination therapy comprising an anti-cancer drug and an anti-Nodal agent, or is a recipient of standard therapy;
(4) the instruction sheet indicates comparing the levels of the ABCA1 and/or Nodal expression before and after the combination therapy or the standard therapy of the cancer patient, and
(5) a negative or a decreased level of the ABCA1 expression after the combination therapy indicates the combination therapy is appropriate and drug resistance to the anti-cancer drug is decreased;
(6) a positive or an increased level of the ABCA1 expression after the standard therapy indicates drug resistance is present and suggests the cancer patient should receive an anti-Nodal agent in combination with anti-cancer drug therapy; and
(7) a positive or an increased level of the Nodal expression after the standard therapy indicates the ABCA1 expression is positive or is increased and drug resistance is present and suggests that the cancer patient should receive an anti-Nodal agent in combination with anti-cancer drug therapy.

Further in another aspect, the invention relates to use of combination agents comprising: (a) an anti-ATP-binding cassette transporter AI (anti-ABCA1) antibody; (b) an anti-Nodal antibody; and (c) optionally an instruction sheet for direction of use thereof in the manufacture of a diagnostic kit for use in prognosis of a cancer patient according to the invention.

Yet in another aspect, the invention relates to a method for prognosis of a cancer patient, comprising:
(a) administering combination therapy comprising an anti-cancer drug and an anti-Nodal agent or standard therapy to the cancer patient;
(b) obtaining a tissue sample from the cancer patient,
(c) detecting and/or measuring the level of the ABCA1 and/or Nodal expression in the tissue sample with an anti-ABCA1 antibody and/or an anti-Nodal antibody; and
(d) comparing the levels of the ABCA1 and/or Nodal expression before and after the combination therapy or standard therapy of the cancer patient; wherein:
(i) a negative or a decreased level of the ABCA1 expression after the combination therapy indicates an increased anti-cancer drug efficacy and a decreased anti-cancer drug resistance;
(ii) a positive or an increased level of the ABCA1 expression after the standard therapy indicates drug resistance is present and suggests that the cancer patient should receive an anti-Nodal agent in combination with anti-cancer drug therapy; and
(iii) a positive or an increased level of the Nodal expression after the standard therapy indicates the ABCA1 expression is positive or is increased and drug resistance is present and suggests that the cancer patient should receive an anti-Nodal agent in combination with anti-cancer drug therapy.

In one embodiment, the method for prognosis of the cancer patient of the invention may further comprise providing the diagnostic kit for use in prognosis of the cancer patient according to the invention.

The invention also related to a method for decreasing, suppressing and/or abrogating manifestation or appearance of cancer drug resistance in a cancer subject in need thereof, comprising:
administering to the cancer subject combination therapeutic agents comprising an anti-cancer drug and an anti-Nodal agent to decrease, suppress and/or abrogate manifestation or appearance of the cancer drug resistance in the cancer subject in need thereof, wherein the cancer subject in need thereof has cancer with heterogeneous tumor cells, in which a subpopulation expresses ATP-binding cassette transporter AI (ABCA1).

In one embodiment, the method further comprises the step of administering to the cancer subject in need thereof an anti-cancer drug.

In another embodiment, administering the pharmaceutical composition comprising the anti-Nodal agent is subsequent to administering the anti-cancer drug, wherein the tumor cell subpopulation is resistant to the anti-cancer drug.

In another embodiment, the step of administering the pharmaceutical composition comprising the anti-Nodal agent is simultaneous with the step of administering the anti-cancer drug, wherein the tumor cell subpopulation is resistant to the anti-cancer drug.

The invention also relates to a method of identifying a cancer patient who is non-responsive to a standard therapy, comprising: performing the method for prognosis of a cancer patient comprising steps (a) to (d) according to the invention; wherein step (a) administers the standard therapy to the cancer patient; and step (d) identifies that the cancer patient as non-responsive to the standard therapy when there is a positive or an increased level of the ABCA1 or the Nodal expression after the standard therapy.

In one embodiment, the tissue sample is a blood sample or a tissue biopsy.

In another embodiment, the standard therapy is combination of therapeutic agents selected from the group consisting of (i), (ii) and (iii):
(i) docetaxel, carboplatin, tratuzumab, pertuzumab (TCHP);
(ii) anthracyclines and taxanes (ACT); and
(iii) docetaxel and cyclophosphamide (TC).

In one embodiment, the method for prognosis of cancer patient may further comprise administering to the cancer patient an anti-Nodal agent in combination with anti-cancer drug therapy.

In another embodiment, the method of identifying a cancer patient who is non-responsive to a standard therapy may further comprise administering to the cancer patient an anti-Nodal agent in combination with anti-cancer drug therapy.

These and other aspects will become apparent from the following description of the preferred embodiment taken in conjunction with the following drawings, although variations and modifications therein may be affected without departing from the spirit and scope of the novel concepts of the disclosure.

The accompanying drawings illustrate one or more embodiments of the invention and, together with the written description, serve to explain the principles of the invention. Wherever possible, the same reference numbers are used throughout the drawings to refer to the same or like elements of an embodiment.

### BRIEF DESCRIPTION OF THE DRAWINGS

FIG. 1 shows the percent of the tumor(s) that expresses Nodal compared to the therapeutic treatment. Clinically derived tissue sections from breast cancer patients, both pre- and post-treatment with current standard-of-care therapy (ACT, TCHP and TC), were examined for the presence of Nodal protein by immunohistochemistry.

FIGS. 2A-D show the results of immunohistochemical staining of Nodal protein in breast cancer patient tumor sections pre- and post-current standard-of-care-treatments. The presence of the Nodal protein (brown color) in breast cancer patient tumor sections pre- and post-current standard-of-care-treatments (ACT, TCHP and TC), was examined by immunohistochemical staining with IgG used as a control for non-specific staining (×20 original magnification). Bar graphs below the IHC data depict the percent of tumor positive for Nodal pre- and post-therapy correlated with the number of lymph nodes involved during the same time frame.

FIGS. 3A-B show that the standard-of-care-treatments used in the study (ACT, TCHP and TC) decreased the size of the tumors but did not change the percent of Nodal in the tumor. (3A) There is a statistically significant decrease in the tumor size in response to the different standard-of-care-treatments (p=0.015, Wilcoxon signed-rank test); while (3B) there is a statistically insignificant change in the percent of Nodal in the tumor after treatment (p=0.27, Wilcoxon signed-rank test).

FIG. 4 shows the presence of the ABCA1 protein (brown color) in breast cancer patient tumor sections pre- and post-current standard-of-care-treatments (ACT, TCHP and TC), being examined by immunohistochemical staining with IgG used as a control for non-specific staining (×20 original magnification).

FIGS. 5A-B show aggressive melanoma cells expressing Nodal and ABCA1 proteins.

FIGS. 6A-B show the results of Western blot analysis of C8161 melanoma cells.

FIG. 7 shows the acute lymphoblastic leukemia cell line REH expressing Nodal protein.

FIG. 8 shows patient clinical characteristics in Table 1.

### DETAILED DESCRIPTION OF THE INVENTION

### Definitions

“An effective amount” refers to the amount of an active agent that is required to confer a therapeutic effect on the treated subject. Effective doses will vary, as recognized by those skilled in the art, depending on routes of administration, excipient usage, and the possibility of co-usage with other therapeutic treatment.

The term “treating” or “treatment” refers to administration of an effective amount of a therapeutic agent to a subject, who has a disease, or a symptom or predisposition toward such a disease, with the purpose to cure, alleviate, relieve, remedy, ameliorate, or minimize the symptoms of it, or the predispositions towards it.

As used here, the term “cancer drug resistance” is interchangeable with chemotherapy resistance. It refers to antineoplastic resistance, which is the ability of cancer cells to survive and grow despite anti-cancer therapies. In some cases, cancers can evolve resistance to multiple drugs, called multiple drug resistance.

As used here, the term “combination therapy” refers to “pharmaceutical combination therapy” that is achieved by separate drugs, or where available, dosage forms that contain more than one active ingredient. Combination therapy involves use of combination therapeutic agents.

As used herein, the term “cancer subject” and “cancer patient” is interchangeable.

Anti-cancer drug may be a chemotherapeutic agent and/or a targeted therapeutic agent.

As used herein, “cancer drug resistance” means “resistance to an anti-cancer drug”.

As used herein, “standard therapy” are interchangeable with “standard-of-care therapy” and refers to use of therapeutic agents such as chemotherapeutic agents, targeted therapeutic agents or any combination thereof. Targeted therapeutic agents may include but not limited to antibodies. One example of standard therapy is a combination of therapeutic agents (chemotherapeutic agents and/or targeted therapeutic agents) selected from the group consisting of (i), (ii) and (iii): (i) docetaxel, carboplatin, tratuzumab, pertuzumab (TCHP); (ii) anthracyclines and taxanes (ACT); and (iii) docetaxel and cyclophosphamide (TC).

The term “chemotherapy” is interchangeable with “cytotoxic chemotherapy”, which is the use of drugs to destroy cancer cells. It usually works by keeping cancer cells from growing, dividing and making more cells. works by killing cancer cells and some normal cells.

As used herein, “a pharmaceutical composition” comprises one or more active ingredients and a pharmaceutically acceptable vehicle. For example, a pharmaceutical composition may comprise an anti-Nodal agent and an acceptable vehicle and/or adjuvant. A pharmaceutical composition may comprise combination therapeutic agents comprising an anti-Nodal agent and an anti-cancer drug.

An anti-Nodal agent may be an anti-Nodal antibody or an anti-nodal compound such as CVM-1125 that diminishes Nodal expression. CVM-1125 is an active metabolite of CVM-1118 (TRX-818; alternative name Foslinanib). Foslinanib is for the treatment of liver cancer and neuroendocrine tumor. Its mechanism of action is angiogenesis inhibitor; apoptosis stimulant, and growth inhibitor.

The “Guidance for Industry and Reviewers Estimating the Safe Starting Dose in Clinical Trials for Therapeutics in Adult Healthy Volunteers” published by the U.S. Department of Health and Human Services Food and Drug Administration discloses “a human equivalent dose” may be obtained by calculations from the following formula:

HED=animal dose in mg/kg×(animal weight in kg/human weight in kg)^{0.33}.

Abbreviations: doxorubicin/cyclophosphamide/taxanes (paclitaxel or docetaxel) (ACT); docetaxel/carboplatin/trastuzumab/pertuzumab (TCHP); docetaxel/cyclophosphamide (TC); hypervariable regions (HVRs); complementarity determining regions (CDRs); three heavy chain HVRs or CDRs (HVRH1 or CDRH1 or H1, HVRH2 or CDRH2 or H2, and HVRH3 or CDRH3 or H3); three light chain CDRs (HVRL1 or CDRL1 or L1, HVRL2 or CDRL2 or L2, and HVRL3 or CDRL3 or L3).

U.S. Pat. No. 9,688,750 discloses an anti-Nodal antibody named 3D1, which is incorporated herein by reference in its entirety. The anti-Nodal antibody 3D1 sequences of the heavy and light chains containing complementary determining regions (CDR or ABR, antigen binding regions) as follows

Light_Chain_A_3D1_CHAIN_SEQUENCE (light chain)
(SEQ ID NO: 1)
DIKMTQSPASLSASVGETVTITCRASGNIHNYLAWYQQKQGKSPQLLVYN
AKTLADGVPSRFSGSGSGTQYSLKINSLQPEDFGSYYCQFIFWSTPHVRC
W DQAGTETEAW
(SEQ ID NO: 4) ABR1(CDRL1):
GNIENYLA (27-34 of SEQ ID NO: 1)
(SEQ ID NO: 5) ABR2(CDRL2):
LLVYNAKTLAD (46-56 of SEQ ID NO: 1)
(SEQ ID NO: 6) ABR3(CDRL3):
QHFWSTPHVRCWDQA (89-103 of SEQ ID NO: 1)
Heavy_Chain_B_3D1_CHAIN_SEQUENCE (heavy chain)
(SEQ ID NO: 2)
VKLVESGGGLVKPGGSLKLSCAASGFTFSSYAMSWVRQTPEKRLEWVAS
ISSGGCTYYPDSVKGRETISRDNARNILYLQMSSLRSEDTAMYYCARGS
MITADGNSLLLCYGLLGSRNXHR
(SEQ ID NO: 7) ABR1 (CDRH1):
FTFSSYAMS (26-34 of SEQ ID NO: 2)
(SEQ ID NO: 8) ABR2(CDRH2):
WVASISSGGCTYY (46-58 of SEQ ID NO: 2)
(SEQ ID NO: 9) ABR3(CDRH3):
ARGSMITADGN (95-105 of SEQ ID NO: 2)
Heavy_Chain_C_3D1_CHAIN_SEQUENCE (heavy chain)
(SEQ ID NO: 3)
KLVESGGGINQPGGSMKLSCVASGFTRNYWMSWVRQSPEKGLEWVAE
IRLKSDNYAARYAESVKGKFTISRDDSKSRLYLQMNSLRAEDTGIYY
CTGIRRFAYWGQGTL
(SEQ ID NO: 10) ABR1(CDRH1):
FTFRNYWMS (26-34 of SEQ ID NO: 3)
(SEQ ID NO: 11) ABR2(CDRH2):
WVAEIRLKSDNYAARY (46-61 of SEQ ID NO: 3
(SEQ ID NO: 12) ABR3(CDRH3):
GIRRFAY (99-105 of SEQ ID NO: 3)

Work from our laboratory and others points to the relevance of Nodal signaling underlying the cancer stem cell (CSC) phenotype, unregulated tumor growth and metastasis, and resistance to standard-of-care therapies; however, no direct linkage has been made regarding the mechanism affecting drug resistance—until now. Furthermore, Nodal is a valuable prognostic biomarker in a variety of cancers associated with the aggressive phenotype, including melanoma, glioblastoma, neuroblastoma, pancreatic cancer, leukemia, and cancers of the breast, prostate, ovary, colon, colorectal and gastric adenocarcinoma.

An indirect connection between Nodal positivity and drug resistance has been demonstrated from the global microarray analysis of aggressive versus non-aggressive melanoma cells—where a greater than nine-fold increase in the expression of ABCA1 is associated with the aggressive melanoma phenotype, but no protein confirmation nor experimental manipulation was conducted. Over the past few decades, there has been a growing body of evidence demonstrating the multidrug resistance properties of ATP-binding cassette proteins, which consists of a large family of integral membrane proteins (reviewed in Gillet et al 2007). Recent data from our laboratory show the first direct correlation between Nodal and ABCA1 expression in metastatic melanoma cells. This observation supports the premise that Nodal-positive tumor cells inherently contain drug resistance characteristics—which provides new insights pertinent to selecting and monitoring patients for therapy targeting Nodal.

Despite noteworthy advances in the field of cancer research, tumor cell heterogeneity remains a formidable challenge in the design and delivery of successful therapies. As scientific studies continue to inform our knowledge base regarding unique tumor cell properties that could represent new targets for therapeutic intervention, our armamentarium for strategic approaches broadens. Particularly insightful has been the finding that aggressive melanoma expresses CSC markers such as Nodal, together with drug resistance-associated markers like CD133 and ABCA1. Certainly, the promise of targeting Nodal in aggressive melanoma is emerging as a viable option to pursue in combination with standard-of-care therapy. In recognizing that aggressive tumors utilize multiple mechanisms to survive, our approach to effectively target melanoma heterogeneity, tumor cell plasticity, and functional adaptation and resistance to current therapies must utilize combinatorial strategies to eliminate relapse, disease progression and metastasis.

### Materials and Methods

### Breast Cancer Patient Samples

Archival formalin-fixed and paraffin-embedded breast tissue sections from 14 patients diagnosed with ductal breast cancer (with varying ER, PR status and HER2 expression) were matched relevant to pre- and post-treatment with standard-of-care therapy (ACT, TCHP or TC), and obtained from the Betty Puskar Breast Care Center, Morgantown, W. Va. Patient tumor sections were de-identified and labelled with numerical codes in accordance with the approved West Virginia University expedited IRB protocol (#1705572966R00) for using de-identified patient samples for research.

### Immunohistochemistry

Four microns thick, formalin fixed, paraffin-embedded tissue sections were prepared, and immunohistochemistry was carried out on a DAKO AutostainerPlus (AgilentTechnologies Inc, Santa Clara, Calif., USA), as previously described. Briefly, following antigen retrieval and blocking steps, sections were incubated with a mouse anti-human Nodal antibody (Abcam, ab55676, 1:300; Cambridge, Mass., USA) or rabbit ABCA1 antibody (Novus Biologicals, NB400-105, 1:200; Centennial Colo., USA) for 60 minutes, followed by biotinylated anti-mouse or anti-rabbit secondary antibody respectively (GM601 and GM608, Biocare Medical, LLC, Concord, Calif., USA). The sections were then treated with streptavidin-horseradish peroxidase (TS125HR, Thermo Scientific Lab Vision) and a brown color developed with 3,3′-diaminobenzidine substrate (TA125QHDX, Thermo Scientific Lab Vision). The sections were then counterstained with hematoxylin (NM-HEM, Biocare Medical, LLC). As a negative control, adjacent serial sections were incubated with ChromPure mouse IgG and ChromPure rabbit IgG (015-000-003 and 011-000-003, Jackson Immunoresearch Labs, West Grove, Pa., USA) at the same concentration as primary antibodies. Staining for Nodal and ABCA1 were analyzed and scored blinded with respect to clinical information.

### Statistical Analyses and Clinical Correlations:

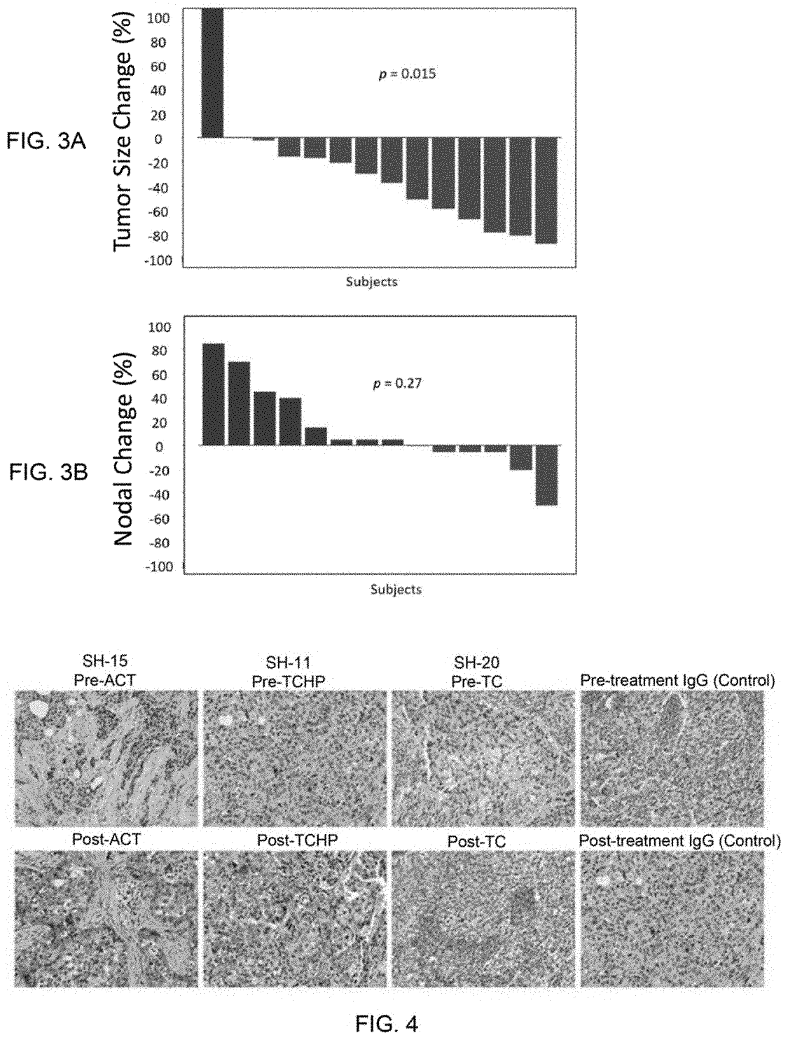

Descriptive statistics was used to summarize data, including frequency distribution and percentage for categorical variables and mean with standard deviation for continuous variables. Bar-plots and waterfall plots were used to demonstrate the data before and after treatment (FIGS. 3A, 3B). In the correlative analysis between treatment and clinical outcomes, Wilcoxon signed-rank test was used to assess the change of nodal and tumor size for the paired data before and after treatment.

### Results and Discussion

A previous, noteworthy breast cancer study from our laboratory focused on Nodal localization in 431 therapeutically naïve patients diagnosed with benign or malignant disease and revealed a potential role for Nodal as a new prognostic biomarker for disease progression, when compared with currently used reference markers. Specifically, the intensity of Nodal IHC staining was significantly stronger (or greater) in undifferentiated, advanced stage invasive breast cancer compared with early stage breast disease. Treatment of human breast cancer cells in vitro with a Nodal blocking antibody results in reduced proliferation and diminished colony-forming ability. Experimental knockdown of Nodal in in vivo models of TNBC also results in significantly reduced levels of tumorigenesis. These findings prompted the translationally relevant question whether Nodal is targeted by standard-of-care therapy in breast cancer patients. Based on the observations derived from our previous studies in melanoma patients and related animal models—where treatment with conventional dacarbazine or BRAF inhibitors did not diminish Nodal expression, we postulated that Nodal would remain before and after standard-of-care therapy in breast cancer patients.

Nodal is Associated with Disease Progression

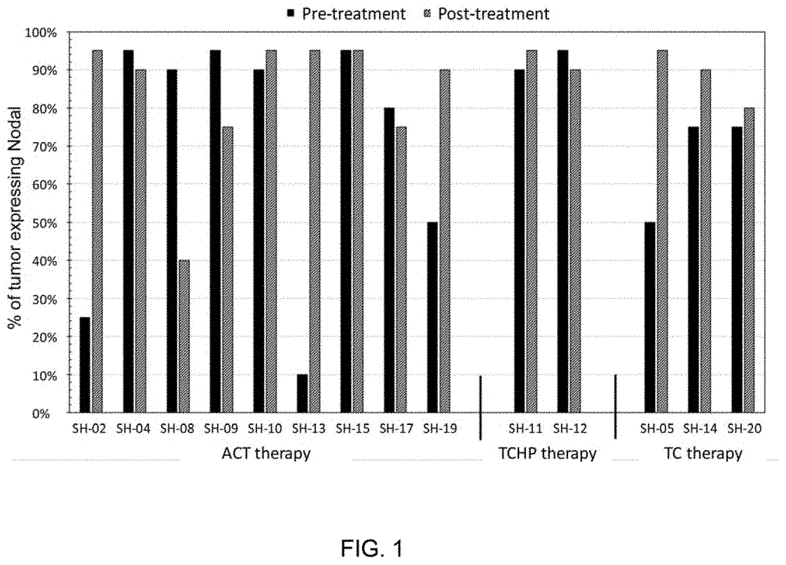

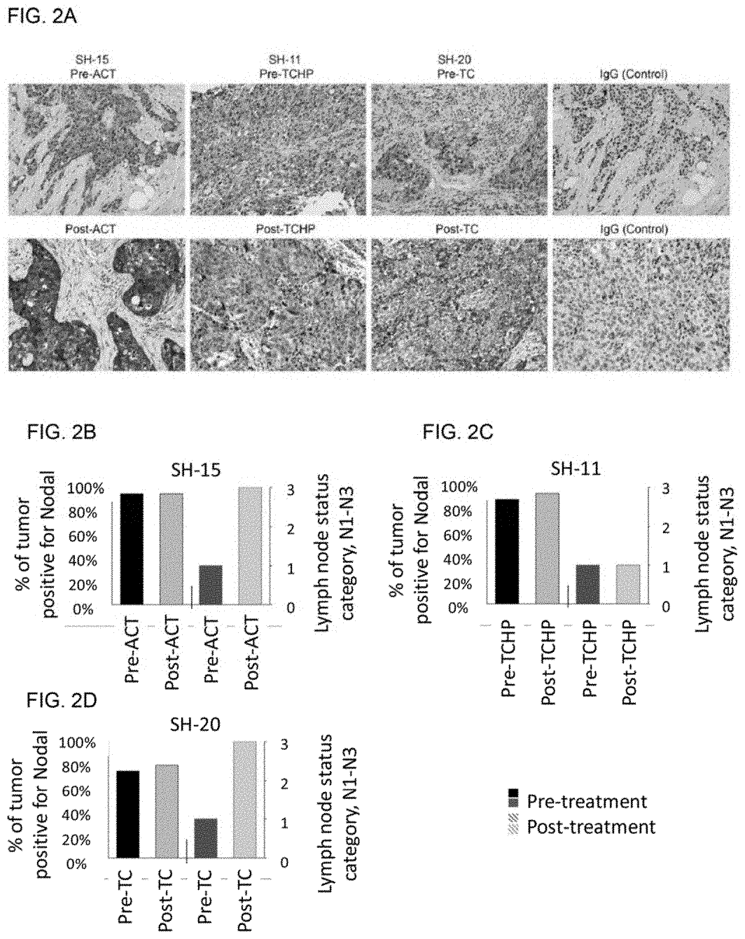

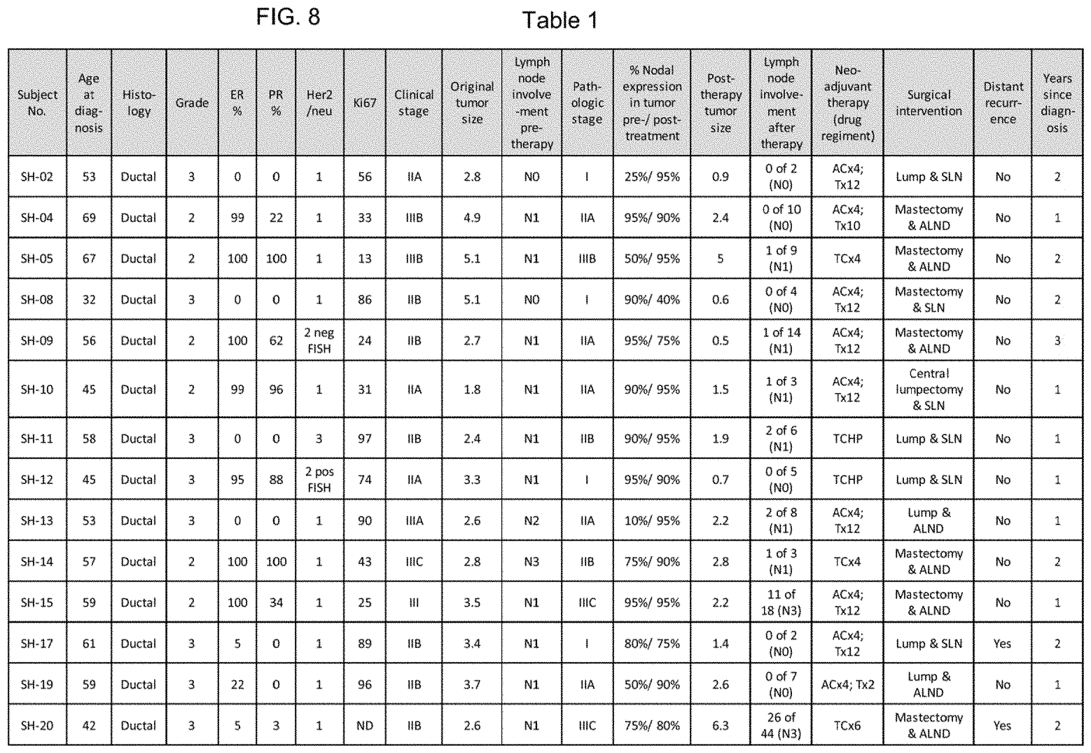

Tissue sections from 14 patients determined to have ductal carcinoma of the breast were studied before and after neoadjuvant therapy using immunohistochemistry (IHC) to evaluate Nodal expression, using a previously established scoring index. Patient clinical characteristics are presented in Table 1 (FIG. 8), including the patients' age, histological description, grade, clinical and pathological stage, original and post-therapy tumor size, status of ER, PR, Her2/neu and Ki67, lymph node involvement before and after therapy, neoadjuvant standard-of-care therapy, surgical intervention, distant recurrence, years since diagnosis, and IHC scores for Nodal pre- and post-treatment. The Nodal IHC analysis of these patients' tumors pre- and post-treatment with current standard-of-care therapy (ACT, TCHP and TC) is presented as a graph in FIG. 1 showing the percent of the tumor that expresses Nodal relevant to the therapeutic treatment. In all cases, Nodal is expressed before and after treatment, and in the majority of cases Nodal expression is enhanced post-treatment. Although a power assessment is not possible to achieve with this small sample number, the data shown support the hypothesis that Nodal is not abrogated by standard-of-care therapy. Nodal IHC staining are presented in FIGS. 2A-D in three patients' tumors where Nodal expression appears enhanced following treatment with ACT, TCHP or TC. Further analysis shows a correlation between enhanced Nodal expression and increased lymph node involvement, supporting the correlation of Nodal expression and disease progression.

FIG. 8 (Table 1) shows specific diagnoses, clinical measurements, treatment regimens and observations for the breast cancer patients' tumors, with incomplete pathological responses, examined in this study.

Nodal is Associated with Drug Resistance

In previous melanoma studies, we observed an association between Nodal expression and the drug resistance marker ABCA1. Most noteworthy, a direct correlation was demonstrated when Nodal expression was down-regulated, resulting in the complete mitigation of ABCA1. These data provided the first direct evidence linking the CSC signaling molecule Nodal and drug resistance, which provided new insights into the possible mechanisms underlying Nodal's role in aggressive cancer. Using tissue sections from the same patient's tumors shown in FIGS. 2A-D for Nodal staining and lymph node involvement, we performed IHC localization for ABCA1 (FIG. 4). The IHC staining pattern for ABCA1 is particularly noteworthy in the post-treatment samples for ACT, TCHP and TC therapy—and may provide new clues linking patient responsiveness relevant to these therapies.

ABCA1 protein is an ATP-binding cassette transporter, which functions as a cholesterol efflux pump in the cellular lipid removal pathway and acts as the primary gatekeeper for eliminating tissue cholesterol. ABCA1 has been shown to be up-regulated in drug resistance to curcumin in melanoma, doxorubicin resistance in breast cancer and hepatocellular carcinoma, paclitaxel and carboplatin-resistance in serous epithelial ovarian cancer, and cisplatin resistance in NCSLC and epidermoid carcinoma. Particularly noteworthy is the Kaplan-Meier plot for ABCA1 low and high expression in breast cancer patient tumors—documented in the NCBI database, which shows a significantly better survival probability for patients expressing low levels of ABCA1 in their breast cancer compared with those who have a high expression.

Collectively, these observations showing a connection between Nodal's presence in aggressive breast cancer—before and after standard-of-care therapy, together with the association with ABCA1 drug resistance marker, provide compelling preliminary evidence to pursue in more depth. Because Nodal acts as a CSC signaling molecule and is expressed by only subpopulations within a heterogeneous tumor, our body of experimental data supports a future therapeutic approach using a standard-of-care therapy in a combinatorial manner with anti-Nodal therapy. We have tested this approach in three experimental TNBC models expressing high levels of Nodal, where treatment with doxorubicin did not effectively diminish the Nodal target. However, sequential treatment of the TNBC models with doxorubicin, followed by anti-Nodal antibody regimen, resulted in significant decreases in cellular growth and viability. This study further revealed that anti-Nodal antibody treatment, following doxorubicin, affects the cellular stress (p38) and repair (ChK1) pathways. These findings support an unique approach in inhibiting Nodal, thereby disrupting the cancer cell's ability to repair their compromised DNA following front-line therapy.

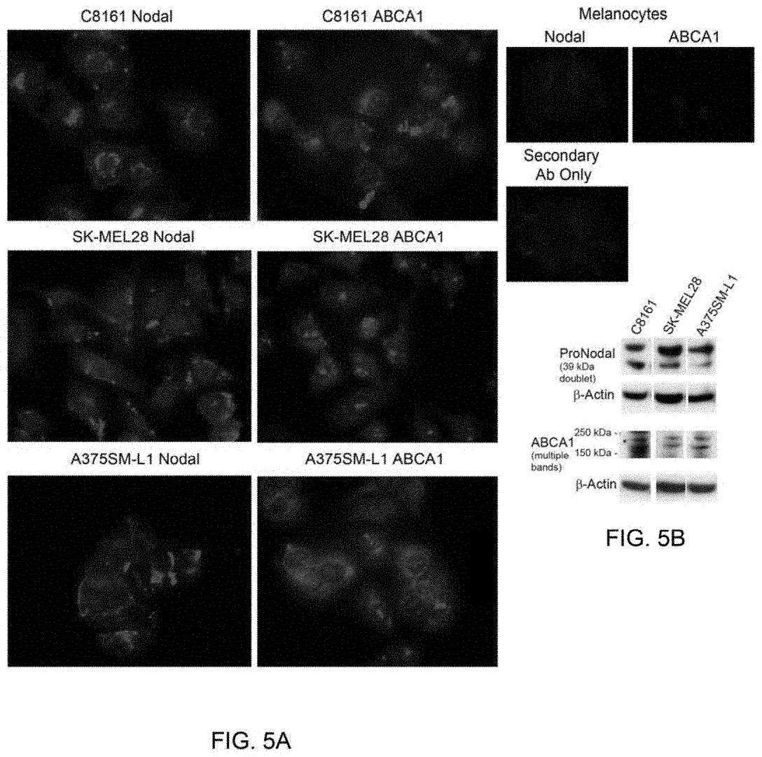

FIGS. 5A-B show that Aggressive melanoma cells express Nodal and ABCA1 proteins. Human metastatic melanoma cell lines C8161, SK-MEL28 and A375SM-L1 and melanocytes were treated with anti-Nodal or anti-ABCA1 antibodies followed by secondary fluorescent antibodies. (A) Cells were viewed using a Zeiss Axioskop 2 microscope and photomicrographs taken comparing immunofluorescence staining of the melanoma cells versus melanocytes (negative control) and cells treated with secondary antibody only (antibody control; magnification 63×). All three aggressive melanoma cell lines express both Nodal and ABCA1 proteins. (B) Furthermore, whole cell protein lysates were prepared from the C8161, SK-MEL28 and A375SM-L1 cells and Western blot analysis verified the expression of both Nodal and ABCA1 proteins at their appropriate molecular weight equivalence. β-Actin protein was used to determine the equal loading of total protein per sample per lane on the Western blot.

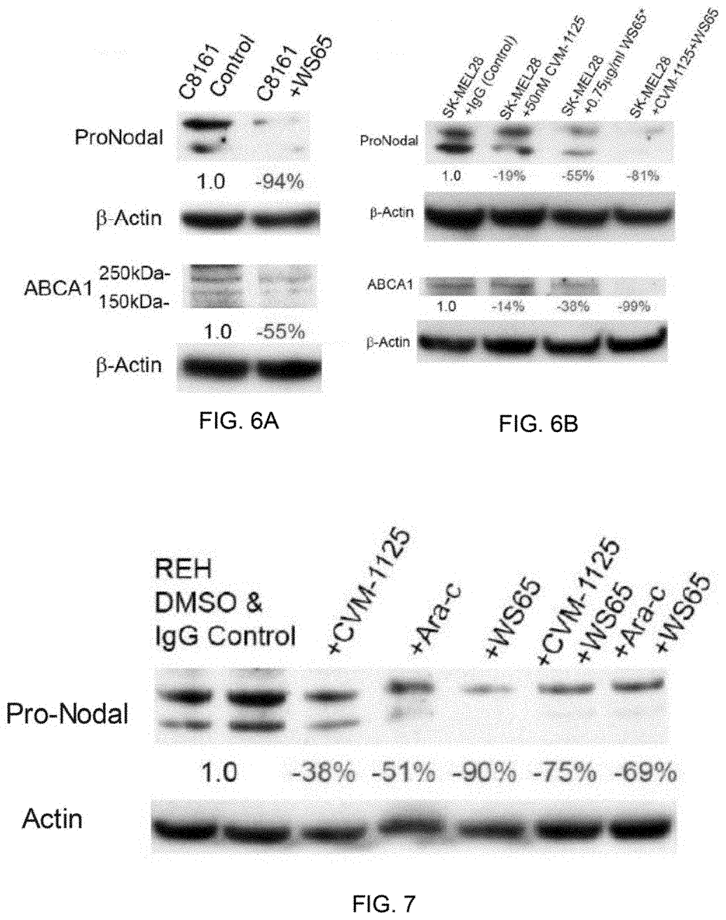

FIGS. 6A-B show that Western blot analysis of C8161 melanoma cells demonstrates that they express Nodal protein (detected in its ProNodal form) and ABCA1 protein. (A) After 72 hr treatment with WS65, an anti-Nodal inhibitory antibody, there is a 94% reduction in Nodal protein expression and 55% reduction in the ABCA1 protein expression. (B) The SK-MEL28 melanoma cell lines also expresses the Nodal and ABCA1 proteins and after treatment CMV-1125 anti-Nodal compound for 72 hours demonstrates a 19% decrease in Nodal protein expression and 14% decrease in ABCA1 protein expression. Similar treatment with WS65 results in a 55% decrease in Nodal protein expression and 38% decrease in ABCA1 protein expression while treatment with both CVM-1125 and WS65 for 72 hr appears to synergistically reduce the expression of Nodal protein by 81% and ABCA1 protein by 99%.

FIG. 7 shows that the acute lymphoblastic leukemia cell line REH expresses Nodal protein (detected as ProNodal on this Western blot). Treatment of the cells for 72 hr with CVM-1125 reduced Nodal protein expression by 38%, while under the same treatment regimen the drug Ara-c reduced Nodal protein expression by 51%, Ws65 reduced Nodal protein by 90%, a combination of CVM-1125 plus WS65 reduced Nodal protein by 75% and Ara-c plus WS65 reduced Nodal protein by 69%.

In aggressive breast cancer, where a lack of targetable molecules exists, together with the likelihood for relapse following chemotherapy, additional studies are needed to evaluate novel biomarkers associated with disease progression and drug resistance. Our discovery of the reactivation of the Nodal signaling pathway in cancer has provided new insights—and instigated additional questions—into the linkage that appears to exist among the CSC phenotype, disease progression, and drug resistance, which can inform the design of more effective clinical trials. Indeed, targeting CSCs and their metastatic niches presents new therapeutic opportunities worth pursuing based on an accumulating body of evidence.

We have learned that normal progenitor cells and CSCs use similar signaling pathways to sustain growth. Moreover, findings related to the convergence of embryonic and tumorigenic signaling pathways have illuminated the significance of oncofetal targets—strictly regulated during normal development, but aberrantly reactivated in aggressive forms of cancer. Particularly noteworthy are oncofetal targets, such as Nodal, that re-emerge only in aggressive cancers but not in normal tissues. The work presented in this paper confirms previous studies showing the importance of Nodal as a CSC molecule associated with aggressive breast cancer—and advances the field by providing new findings indicating that Nodal is not targeted by standard-of-care therapy in breast cancer patients. Most noteworthy is the linkage between Nodal expression and the drug resistance marker ABCA1. Although the results are based on a small sample number, the preliminary findings are of special interest in the design of new therapeutic strategies that target the stem cell properties of adult cancer cells, especially as part of a combinatorial approach to overcome drug resistance and disease recurrence.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments and examples were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

What is claimed is:

1. A method for decreasing, suppressing and/or abrogating manifestation or appearance of cancer drug resistance in a cancer subject in need thereof, comprising:
administering to the cancer subject combination therapeutic agents comprising an anti-cancer drug and an anti-Nodal agent to decrease, suppress and/or abrogate manifestation or appearance of the cancer drug resistance in the cancer subject in need thereof, wherein the cancer subject in need thereof has cancer with heterogeneous tumor cells, in which a subpopulation expresses ATP-binding cassette transporter AI (ABCA1).

2. The method of claim 1, wherein the anti-Nodal agent is selected from the group consisting of an anti-Nodal antibody and CVM-1125.

3. The method of claim 1, wherein the tumor cell subpopulation expresses Nodal in addition to expressing the ABCA1.

4. The method of claim 1, wherein the tumor cell subpopulation is resistant to the anti-cancer drug.

5. The method of claim 1, wherein the cancer is metastatic cancer.

6. The method of claim 1, wherein the anti-cancer drug is a combination of therapeutic agents selected from the group consisting of (i), (ii) and (iii):
(i) docetaxel, carboplatin, tratuzumab, pertuzumab (TCHP);
(ii) anthracyclines and taxanes (ACT); and
(iii) docetaxel and cyclophosphamide (TC).

7. A method for decreasing, suppressing and/or abrogating manifestation or appearance of cancer drug resistance in a cancer subject in need thereof, comprising the step of:
administering to the cancer subject in need thereof a pharmaceutical composition comprising an anti-Nodal agent, wherein the cancer subject in need thereof has cancer with heterogeneous tumor cells comprising a subpopulation that expresses ATP-binding cassette transporter AI (ABCA1).

8. The method of claim 5, wherein the pharmaceutical composition comprises combination therapeutic agents comprising the anti-Nodal agent and an anti-cancer drug.

9. The method of claim 7, wherein the cancer is selected from the group consisting of breast cancer, melanoma, prostate cancer, pancreatic cancer and ovarian cancer.

10. The method of claim 7, wherein the cancer subject in need thereof is a recipient of an anti-cancer drug.

11. The method of claim 7, further comprising the step of:
administering to the cancer subject in need thereof an anti-cancer drug.

12. The method of claim 11, wherein administering the pharmaceutical composition comprising the anti-Nodal agent is subsequent to administering the anti-cancer drug, wherein the tumor cell subpopulation is resistant to the anti-cancer drug.

13. The method of claim 1, wherein the step of administering the pharmaceutical composition comprising the anti-Nodal agent is simultaneous with the step of administering the anti-cancer drug, wherein the tumor cell subpopulation is resistant to the anti-cancer drug.

14. The method of claim 8, wherein the cancer is metastatic cancer.

15. The method of claim 8, wherein the anti-cancer drug is a combination of therapeutic agents selected from the group consisting of (i), (ii) and (iii):
(i) docetaxel, carboplatin, tratuzumab, pertuzumab (TCHP);
(ii) anthracyclines and taxanes (ACT); and
(iii) docetaxel and cyclophosphamide (TC).

16. A diagnostic kit for use in prognosis of a cancer patient, the diagnostic kit comprising:
(a) an anti-ATP-binding cassette transporter AI (anti-ABCA1) antibody;
(b) an anti-Nodal antibody; and
(c) an instruction sheet for direction of use thereof;
wherein:
(1) the anti-ABCA1 antibody is for use in detecting and/or measuring the level of ATP-binding cassette transporter AI (ABCA1) expression in a tissue sample from the cancer patient;
(2) the anti-Nodal antibody is for use in detecting and/or measuring the level of Nodal in a tissue sample from the cancer patient;
(3) the cancer patient is a recipient of combination therapy comprising an anti-cancer drug and an anti-Nodal agent, or is a recipient of standard chemotherapy;
(4) the instruction sheet indicates comparing the levels of the ABCA1 and/or Nodal expression before and after the combination therapy or the standard therapy of the cancer patient, and
(5) a negative or a decreased level of the ABCA1 expression after the combination therapy indicates the combination therapy is appropriate and drug resistance to the anti-cancer drug is decreased;
(6) a positive or an increased level of the ABCA1 expression after the standard therapy indicates drug resistance is present and suggests the cancer patient should receive an anti-Nodal agent in combination with anti-cancer drug therapy; and
(7) a positive or an increased level of the Nodal expression after the standard therapy indicates the ABCA1 expression is positive or is increased and drug resistance is present and suggests that the cancer patient should receive an anti-Nodal agent in combination with anti-cancer drug therapy.

17. A method for prognosis of a cancer patient, comprising:
(a′) providing the diagnostic kit of claim 16;
(a) administering combination therapy comprising an anti-cancer drug and an anti-Nodal agent or standard therapy to the cancer patient;
(b) obtaining a tissue sample from the cancer patient;
(c) detecting and/or measuring the level of the ABCA1 and/or Nodal expression in the tissue sample with an anti-ABCA1 antibody and/or an anti-Nodal antibody; and
(d) comparing the levels of the ABCA1 and/or Nodal expression before and after the combination therapy or standard therapy of the cancer patient; wherein:
(i) a negative or a decreased level of the ABCA1 expression after the combination therapy indicates an increased anti-cancer drug efficacy and a decreased anti-cancer drug resistance;
(ii) a positive or an increased level of the ABCA1 expression after the standard therapy indicates drug resistance is present and suggests that the cancer patient should receive an anti-Nodal agent in combination with anti-cancer drug therapy; and
(iii) a positive or an increased level of the Nodal expression after the standard therapy indicates the ABCA1 expression is positive or is increased and drug resistance is present and suggests that the cancer patient should receive an anti-Nodal agent in combination with anti-cancer drug therapy.

18. A method of identifying a cancer patient who is non-responsive to a standard therapy, comprising:
performing the method of claim 17; wherein step (a) administers the standard therapy to the cancer patient; and step (d) identifies that the cancer patient as non-responsive to the standard therapy when there is a positive or an increased level of the ABCA1 or the Nodal expression after the standard therapy.

19. The method of claim 17, wherein the tissue sample is a blood sample or a tissue biopsy.

20. The method of claim 17, wherein the standard therapy is combination of therapeutic agents selected from the group consisting of (i), (ii) and (iii):
(i) docetaxel, carboplatin, tratuzumab, pertuzumab (TCHP);
(ii) anthracyclines and taxanes (ACT); and
(iii) docetaxel and cyclophosphamide (TC).
